(19) United States
(12) Patent Application Publication
(10) Pub. No.: US 2006/0008534 A1
(43) Pub. Date: Jan. 12, 2006

(54) Crystal liquid/crystal bioenergy device

(76) Inventor: Francis Lyle Jordan, Boise, ID (US)

Correspondence Address: Frank Jordan, 723 W. Victory Road, Boise, ID, 83706, US

(21) Appl. No.: 10/888,935

(22) Filed: Jul. 12, 2004

(51) Int. Cl. A61K 33/24; A61K 33/00

(52) U.S. Cl. 424/617; 424/724

(57) ABSTRACT

My invention capitalizes on the fact that a crystal in resonance with a liquid such as water in a tube or closed container, acts to either pattern the liquid to a desired set of frequencies, or to act as an electromagnetic attractant to assimilate and clear adverse energies from the energy fields of the body. Further, with my invention, a person may visualize, or energetically desire, a beneficial condition and transfer that pattern of energy into a crystal, which in turn holds the pattern and transmits it to a liquid, transforming the liquid into a liquid crystal that resonates with the water in the blood, the DNA, and the vital life forces. This energetic reaction has long been used in energetic medicine, and through flower essences and other agents that effect positive physiological and psychological responses, as well as through use of drugs, prescriptions, and remedies. All medical and holistic health practitioners rely upon the allopathic effects of such responses to stimulate cellular consciousness to regain a balanced state. Further, such attunement between consciousness, DNA, and vital life forces through the crystal liquid/crystal connection may allow a direct communication between the energetic intelligence frequencies of the mind and other crystal based intelligences such as humans and artificial intelligent systems such as found in computers.

### REFERENCES CITED (U.S. PATENT DOCUMENTS)

Sabuda, Thomas J. U.S. Pat. No. 6,048,301 Apr. 11, 2000 “Method and Device For Stimulating Biological processes”

Van Dick, Robert C. U.S. Pat. No. 5,562,597 Oct. 8, 1996, “Method and Apparatus for reducing Psychological Stress.”

Bradish, Et Al U.S. Pat. No. 5,830,064 Nov. 3, 1998 “Apparatus and method for distinguishing events which collectively exceed chance expectations and thereby controlling an output.”

### BACKGROUND OF INVENTION DESCRIPTION

This invention relates generally to the use of quartz or other crystals to resonate with a liquid solution, water or otherwise, to effect alterations or clearing of energy systems within the body, or the energy fields surrounding the body, or the environment of the body. Further, such crystal/liquid crystal resonance may allow direct energetic attunement of the subtle energies of consciousness, mind, and Spirit with other crystal based memory units such as other humans and computers. Biologists are beginning to understand that many substances and membranes in the human body act or function as liquid crystals, a new class of crystals. Bio-crystals have structures that are partly crystalline and part liquid, depending on their location and function. Bio-energy liquid crystals are found to be involved in the attunement of subtle energies of the nervous system and the life-force systems of the body.

Between the, subtle energy of consciousness and life force system of the body, electromagnetic current is created by the polarity difference between them. Crystalline substances operate on sympathetic resonance, and attunement between the life force and subtle conscious is necessary for a balanced energy system. A balanced energetic system in the mind/body/Spirit connections is necessary for good health, vitality, and enjoyment of life. A crystal liquid/crystal device, when properly programmed, may be used to clear toxicity and negative electromagnetic energies and thoughtforms adversely affecting health and mental well-being.

My invention capitalizes on the fact that a crystal in resonance with a liquid crystal such as water, acts to either pattern the liquid to a desired set of frequencies, or to act as an electromagnetic attractant to assimilate and clear adverse energies from the energy fields of the body.

Further, with my invention, a person may visualize, or energetically desire, a beneficial condition and transfer that pattern of energy into a crystal, which in turn holds the pattern and transmits it to a liquid, transforming the liquid into a liquid crystal that resonates with the water in the blood, the DNA, and the vital life forces. This energetic reaction has long been used in energetic medicine, and through flower essences and other agents that effect positive physiological and psychological responses, as well as through use of drugs, prescriptions, and remedies. All medical and holistic health practitioners rely upon the allopathic effects of such responses to stimulate cellular consciousness to regain a balanced state.

Further, such attunement between consciousness, DNA, and vital life forces through the crystal liquid/crystal connection may allow a direct communication between the energetic intelligence frequencies of the mind and other crystal based intelligences such as humans and artificial intelligent systems such as found in computers.

### DESCRIPTION OF PREFERRED EMBODIMENTS

The preferred embodiment of this invention is a tube, or other container, plastic or otherwise, that contains water, or any other solution. A crystal, quartz, or otherwise, is introduced into the solution, or near proximity, to act as the resonating device. For example, a plastic tube may have a crystal included in either or both ends, with a stopper in both ends to contain the water, and to extract air bubbles from the system. This embodiment would have a positive right end, and a negative left end, depending upon the orientation of the included crystals.

In this case, the crystal in the left end would be oriented with the positive end pointing toward the center of the tube. On the right end of the tube, the positive point of the crystal would point away from the center of the tube, and away from the body. In use, the person would hold the negative, or left end of the tube, in his left hand, and the positive, or right end in his right hand. The tube would be of sufficient length to wrap around the body, allowing the liquid crystal to interact with the energy systems of the body, and the etheric energy fields surrounding it. Because of the polarity orientation of the tube, negative, or disharmonious energy, such as stress, would channel the length of the tube, and exit from the positive tip of the right hand crystal, into the earth energy polarity grid system.

In other embodiments, ground crystals may be included in the liquid crystal water matrix to enhance energy transfer, or to impart specific frequencies of vibration.

In other embodiments, energized tubes could be used to surround environmental locations, such as beds, to clear energy fields released in sleep, or to program a beneficial environment, and to block noxious energies.

In other embodiments, containers, or witness chambers, could be included in each end of the tube, into which crystals of specific frequency or programming could be introduced to pattern the liquid crystal, or hold desired frequencies.

In other embodiments, a liquid crystal/crystal tube could be placed in, around, or in close proximity, to a computer or other artificial intelligence device employing a crystal based matrix, to allow direct communication of subtle energies of consciousness with such a device.

These embodiments are included as examples only, and are not to exclude any other embodiment that utilizes a crystal to pattern or charge a liquid crystal, or any potential use thereof.

### SUMMERY OF THE INVENTION

As has been summarized in the previous discussion, Holistic practitioners, Shaman, Alternative healing practices, and conventional medicine have long recognized crystals as possessing properties that can be employed to enhance healing, and for clearing negative thought forms and energy fields from the body. Biophysicists recognize that water and other solutions are liquid crystals. The body contains crystalline substances that resonate with the body fluids, giving us the opportunity to employ crystals for direct intervention in the energy fields of the body, mind and consciousness. Through resonance, Desire and Will of consciousness can be employed to install beneficial patterns into the energy systems of the body, or to directly establish resonance of consciousness between subtle energy systems of the mind/Spirit/body and systems of artificial intelligence.

1. The union of a crystal with a liquid imparts qualities of the crystal to the liquid, creating through resonance, a liquid crystal.

2. A liquid crystal may resonate with the energy systems of the mind and body and the energy system surrounding the body.

3. Disharmonious energies may be channeled from the body, utilizing polarities instilled in the liquid crystal by the crystal.

4. The liquid crystal may balance, or clear, energies inharmonious to the body from the environment.

5. A crystal may be patterned with frequencies, which impart to a liquid crystal desirable characteristics, which in turn resonate with the liquid crystal in the body to pattern beneficial conditions in the body.

6. The liquid crystal/crystal resonance can be used to clear stress from the body, to enhance meditation and deep relaxation.

7. Specific emotional conditions, such as peace and security, and other desired expressions, can be patterned into the crystal liquid/crystal device, and imparted into the body through the energetic grid of consciousness surrounding the body.

8. Resonance between life forces, consciousness, mind, body, may be amplified and enhanced with the use of the crystal/liquid crystal device.

9. The crystal liquid/crystal resonance may be useful to amplify resonance between people to enhance psychic impression, telepathic communication, distant healing, and other subtle energy interactions through the consciousness grind

10. The crystal liquid/crystal device may be used to enhance clearing, stabilization, and patterning of the various levels of conscious system with the body known as the Chakra system

11. The crystal liquid/crystal device can be used to clear inharmonious energy patterns between people to enhance relationship.

12. The crystal liquid/crystal can be used to clear undesirable personal programs, and to store and intensify desired programs in the mind/body connection, such as weight loss, healing, mood, habit alteration, and compulsive or addictive behavior.
